(19) United States
(12) Patent Application Publication
(10) Pub. No.: US 2025/0049784 A1
(43) Pub. Date: Feb. 13, 2025

(54) METHODS OF TREATING PROSTATE CANCER OR METASTASIZED PROSTATE CANCER

(71) Applicant: Zetagen Therapeutics, Inc., Syracuse, NY (US)

(72) Inventor: Bryan S. Margulies, Liverpool, NY (US)

(73) Assignee: Zetagen Therapeutics, Inc., Syracuse, NY (US)

(21) Appl. No.: 18/366,439

(22) Filed: Aug. 7, 2023

(51) Int. Cl. A61K 31/485; A61K 47/02; A61K 47/42; A61P 35/00

(52) U.S. Cl. CPC A61K 31/485; A61K 47/02; A61K 47/42; A61P 35/00

(57) ABSTRACT

A composition is provided for treating a cancer which includes a high concentration of an OGFR antagonist in a formulation that may also include collagen and calcium phosphate. The composition is useful for treating a cancer when locally administered at a site of cancer in a patient.

### SEQUENCE LISTING

The instant application contains a Sequence Listing which has been submitted electronically in XML format and is hereby incorporated by reference in its entirety. Said XML copy, created on Jul. 24, 2023, is named 118922-0251_SL.xml and is 5,600 bytes in size.

### FIELD

The present disclosure relates in general to pharmaceutical formulations, their uses for treating diseases and their methods of making and more specifically to pharmaceutical formulations comprising an Opioid Growth Factor Receptor (OGFR) antagonist, uses for treating diseases and their methods of making.

### SUMMARY

One embodiment is a pharmaceutical formulation comprising an opioid growth factor receptor (OGFR) antagonist and a pharmaceutically acceptable carrier comprising calcium phosphate and collagen. Preferably, the formulation comprises a powder component and a liquid component (the liquid component comprising at least one carrier and an effective amount of the OGFR antagonist), which are mixed together prior to administration. Preferably, the resulting formulation is administered by injection to a cancer patient.

Another embodiment is a method of treating a cancer comprising administering to a subject in need thereof a therapeutically effective amount of a pharmaceutical formulation comprising an opioid growth factor receptor (OGFR) antagonist and a pharmaceutically acceptable carrier comprising calcium phosphate and collagen.

Another embodiment is a kit for treating cancer comprising a powder component and a liquid component, wherein the liquid component comprises at least one pharmaceutically acceptable carrier and an effective amount of the OGFR antagonist. Preferably the kit further includes instructions for use, including instructions for mixing the powder component and liquid component together prior to administration and using the resulting formulation to treat cancer.

### FIGURES

FIG. 1 shows change in cell number following treatment of PC3 cells with ZP001. Non-linear regression curves were computed for the PC3 prostate cancer cells at 24-hours (black line) and 72-hours (gray line). The regression lines were shown to be significantly different via an Extra-Sum-of-Squares F-test (p<0.0001), which means the data is best represented as separate lines (i.e., separate populations). At specific time points, only significant differences in PC3 cell number were observed for the 5-mM concentration (*=p<0.012) and the 10-mM concentration (**=p<0.0007).

FIG. 2 shows LDH % cytotoxicity in PC3 cells following treatment with ZP001.

FIG. 3 shows multicellular tumor spheroid area 2-weeks after administration of ZP001.

FIG. 4 shows tumor volume in mice inoculated with the PC3 prostate cancer cells and treated with ZP001 2-weeks later and 25-days later.

### DETAILED DESCRIPTION

### Definitions

The following definitions are provided to facilitate understanding of certain terms used throughout this specification.

Technical and scientific terms used herein have the meanings commonly understood by one of ordinary skill in the art, unless otherwise defined. Any suitable materials and/or methodologies known to those of ordinary skill in the art can be utilized in carrying out the methods described herein.

As used in the description of the invention and the appended claims, the singular forms “a”, “an” and “the” are used interchangeably and intended to include the plural forms as well and fall within each meaning, unless the context clearly indicates otherwise. Also, as used herein, “and/or” refers to and encompasses any and all possible combinations of one or more of the listed items, as well as the lack of combinations when interpreted in the alternative (“or”).

All numerical designations, e.g., pH, temperature, time, concentration, amounts, and molecular weight, including ranges, are approximations which are varied (+) or (−) by 10%, 1%, or 0.1%, as appropriate. It is to be understood, although not always explicitly stated, that all numerical designations may be preceded by the term “about.” It is also to be understood, although not always explicitly stated, that the reagents described herein are merely exemplary and that equivalents of such are known in the art.

The term “comprising” or “comprises” is intended to mean that the compositions and methods include the recited elements, but do not exclude others. “Consisting essentially of,” when used to define compositions and methods, shall mean excluding other elements of any essential significance to the combination. For example, a composition consisting essentially of the elements as defined herein would not exclude other elements that do not materially affect the basic and novel characteristic(s) of the claimed invention. “Consisting of” shall mean excluding more than trace amount of other the ingredients and substantial method steps recited by the claims. Embodiments defined by each of these transition terms are within the scope of this invention.

As used here, the term “antagonist” is used interchangeably with “inhibitor” and refers to a substrate that blocks or suppresses the activity, function, effect, or expression of a target. In some embodiments, the target is a compound, a protein, a gene, a cell, or an agent. As used herein, the term “expression” refers to the amount a living cell produces of a target. In some embodiments, the inhibitor suppresses expression of a target gene or protein. In some embodiments, the inhibitor includes a compound that prevents binding of another molecule to an enzyme or molecular pump. In some embodiments, the inhibitor is a compound that causes downregulation of the enzyme. In some embodiments, the inhibitor can be a competing or non-competing inhibitor.

The term “administering” as used herein includes prescribing for administration as well as actually administering, and includes physically administering by the subject being treated or by another. As used herein “subject,” “patient,” or “individual” refers to any subject, patient, or individual, and the terms are used interchangeably herein. In this regard, the terms “subject,” “patient,” and “individual” includes mammals, and, in particular humans. When used in conjunction with “in need thereof,” the term “subject,” “patient,” or “individual” intends any subject, patient, or individual having or at risk for a specified symptom or disorder.

As used herein, the phrase “therapeutically effective” or “effective” in context of a “dose” or “amount” means a dose or amount that provides the specific pharmacological effect for which the compound or compounds are being administered. It is emphasized that a therapeutically effective amount will not always be effective in achieving the intended effect in a given subject, even though such dose is deemed to be a therapeutically effective amount by those of skill in the art. For convenience only, exemplary dosages are provided herein. Those skilled in the art can adjust such amounts in accordance with the methods disclosed herein to treat a specific subject suffering from a specified symptom or disorder. The therapeutically effective amount may vary based on the route of administration and dosage form.

The term “treating” or “treatment” covers the treatment of a cancer described herein, in a subject, such as a human, and includes (i) inhibiting a cancer, i.e., arresting its development; (ii) relieving a cancer or disorder, i.e., causing regression of the cancer; (iii) slowing progression of the cancer; and/or (iv) inhibiting, relieving, or slowing progression of one or more symptoms of the cancer. For example, treatment of a cancer includes, but is not limited to, elimination of the cancer or the condition caused by the cancer, remission of the tumor, inhibition of the cancer, or reduction or elimination of at least one symptom of the tumor.

The term “analog” refers to a compound in which one or more individual atoms or functional groups have been replaced, either with a different atom or a different functional group, generally giving rise to a compound with similar properties. In some aspect, the analog refers to a structure that is similar to another but differs in one or two components.

The term “derivative” refers to a compound that is formed from a similar beginning compound by attaching another molecule or atom to the beginning compound. Further, derivatives, according to the invention, encompass one or more compounds formed from a precursor compound through addition of one or more atoms or molecules or through combining two or more precursor compounds.

As used herein, the terms “resorbable” and “bioresorbable” refers to the capability of a material to be broken down over a period of time and assimilated into the biological environment. Resorbable and bioresorbable, in the context of a human body environment, implies that the material is broken down over a period of time and assimilated into the body under normal physiological conditions.

As used herein, the term “moldable” refers to the property of being pliable, able to be compressed, shaped, and manipulated by force of hand, while maintaining integrity, homogeneity of the composition, physical properties, and performance properties.

As used herein, references to a weight of components of a bone graft composition or material described herein, such as the phrase “by weight,” refer to the weight of the applicable component prior to being added to or mixed with another different component of the bone graft composition. For example, the weight can refer to an initial weight of the component measured out before further processing of the component into the bone graft composition.

### Opioid Growth Factor Receptor (OGFR) Antagonists

“Opioid Growth Factor Receptor (OGFR) antagonist” may mean a molecule that inhibits, suppresses and/or causes the cessation of at least one OGFR-mediated biological activity. One example of OGFR antagonist may be naloxone.

In some embodiments, an OGFR antagonist is an OGFR binding antagonist, namely, a molecule that, interferes with, blocks or otherwise prevents the interaction or binding of the met5-ligand (OGF) to the OGFR. Met-5 is derived from the pro-hormone pro-enkephalin (PENK).

An OGFR binding antagonist may compete with the met5-ligand for binding to the OGFR on the surface of the nuclear membrane, thereby interfering with, blocking or otherwise preventing the binding of the met5-ligand to the OGFR, without triggering the downstream signaling that would otherwise be induced by the binding of the met5-ligand to the OGFR. Alternatively, an OGFR binding antagonist may bind to or sequester pro-enkephalin (PENK) or the met5-ligand with sufficient affinity and specificity to substantially interfere with, block or otherwise prevent binding of met5-ligand to the OGFR, thereby inhibiting, suppressing or causing the cessation of at least one OGFR-mediated biological activity. Generally speaking, OGFR binding antagonists may be large molecules (e.g., antibodies) or small molecules (e.g., compounds of a molecular weight of less than 15-kD, 12-kD, 10-kD or even 8-kD), and may be a polypeptide, nucleic acid, or a synthetic small molecule compound. OGFR binding antagonists may be identified with any in vitro assay readily selected by one of skill in the art. For example, OGFR antagonists may be identified using the methods described in Zagon et al., Brain Research Reviews, 2002, 38 (3): 351-76. Other suitable OGFR antagonists are disclosed in PCT patent application publications Nos. WO2021/011529; WO2022/015364; U.S. patent application publications Nos. 2019-0093109; 2021-0030746; 2021-0228571; 2022-0016312; and U.S. Pat. No. 11,471,454.

In some embodiments, the OGFR binding antagonist is naloxone or a functional derivative thereof, naltrexone or a functional derivative thereof, or a combination thereof.

As used herein, a “functional derivative” refers to a derivative or analog that is structurally and functionally analogous to the originating molecule (e.g., maintains the function of naltrexone or naloxone as an OGFR antagonist). Naloxone and naltrexone analogs can be synthesized using standard synthetic procedures such as those described in March J., Advanced Organic Chemistry, 3rd Ed. (1985). Examples of naltrexone and naloxone functional derivatives include salt forms, e.g., naloxone hydrochloride dihydrate or naltrexone hydrochloride. Additional examples of naltrexone and naloxone functional derivatives suitable for use in the present methods include naltrexone and naloxone analogs disclosed in U.S. Patent Application Publication No. 2007/0197573 A1, U.S. Pat. No. 6,713,488, for example.

In some embodiments, an OGFR binding antagonist may be derived from oxymorphone and binds to the OGFR, which includes naloxone, naltrexone, nalorphine, naloxonazine, levallorphan, nalmefene, cyprodime, cyclorphan, cyclazocine, oxilorphan, LY113878, MR2266, diprenorphine, WIN 44,441-3, naltindole, or norbinaltorphimine.

In still another embodiment, an OGFR binding antagonist may be derived from trans-3,4-dimethyl-4-phenylpiperidine and binds to the OGFR, which includes LY99335, LY25506, LY117413, or LY255582. In another embodiment, an OGFR binding antagonist is derived from the met5-enkephalin or leu-enkephalin peptides, binds to the OGFR, and minimally includes the following amino acid sequences as a means of targeting the OGFR: Tyr-Gly-Gly-Phe-Met (SEQ ID NO: 1) for those derived from met5-enkephalin or Tyr-Gly-Gly-Phe-Leu (SEQ ID NO: 2) for those derived from the leu-enkephalin. In still another embodiment, an OGFR binding antagonist is derived from the peptide antagonist 101174864 (N,N-diallyl-Tyr-Aib-Aib-Phe-Leu-OH; Aib-aminoisobutyticacid) or somatostatin analog CTP(D-Phe-Cys-Tyr-D-Trp-Lys-Thr-Pen-Thr-NH.sub.2, SEQ ID NO: 3).

In other embodiments, the OGFR antagonist, instead of being an OGFR binding antagonist, is a molecule that disrupts the nuclear localization sequence found within OGFR: 251

QSALDYFMFAVRCRHQRRQLVHFAWEHFRPRCKFVWGPQDKLRRFKPSSL (SEQ ID NO: 4).

In still other embodiments, the OGFR antagonist employed in the present methods is a small-hairpin RNA (shRNA) or a small-interfering RNA (siRNA) directed against the OGFR gene and effective in disrupting OGFR gene expression.

The OGFR antagonists described herein may be administered individually or in combination. Suitable combinations include, for example, naloxone and naltrexone; naloxone and/or naltrexone, in combination with another OGFR binding antagonist or another OGFR antagonist.

### DISCLOSURE

The present disclosure is based on a discovery that a higher concentration of an OGFR antagonist, such as naloxone, may be achieved in a formulation, which includes calcium phosphate and a collagen as pharmaceutically acceptable carriers compared to formulations of the OGFR antagonist with carriers which do not include the combination of calcium phosphate and a collagen.

Thus, one embodiment is a pharmaceutical formulation comprising an opioid growth factor receptor (OGFR) antagonist, such as naloxone, and a pharmaceutically acceptable carrier comprising calcium phosphate and collagen.

A concentration of the opioid growth factor receptor (OGFR) antagonist, such as naloxone, in the formulation may be at least 1-mM, at least 2-mM, at least 3-mM, at least 4-mM, at least 5-mM, at least 6-mM, at least 7-mM, at least 8-mM, at least 9-mM, at least 10-mM, at least 11-mM, at least 12-mM, at least 13-mM, at least 14-mM, at least 15-mM, at least 16-mM, at least 17-mM, at least 18-mM, at least 19-mM, at least 20-mM or at least 21-mM.

For example, in some embodiments, a concentration of naloxone in the formulation may be at least 0.4-mg/ml, at least 0.8-mg/ml, at least 1.2-mg/ml, at least 1.6-mg/ml, at least 2.0-mg/ml, at least 2.4-mg/ml, at least 2.8-mg/ml, at least 3.2-mg/ml, at least 3.6-mg/ml, at least 4.0-mg/ml, at least 4.4-mg/ml, at least 4.8-mg/ml, at least 5.2-mg/ml, at least 5.6-mg/ml, at least 6.0-mg/ml, at least 6.4-mg/ml, at least 6.8-mg/ml, at least 7.2-mg/ml, at least 7.6-mg/ml, at least 8.0-mg/ml, at least 8.4-mg/ml, at least 8.8-mg/ml, at least 9.2-mg/ml, at least 9.4-mg/ml, at least 9.8-mg/ml, at least 10.2-mg/ml, at least 10.6-mg/ml, at least 11.0-mg/ml, or at least 11.4-mg/ml.

The present formulation may be used for treating a cancer. In some embodiments, the present formulation may be used for treating a cancer, for which a lower concentration of naloxone, such as 1-mM or 0.4-mg/ml, may not be effective. One example of such cancer may be a prostate cancer or other slow growing cancers, such as meningiomas and oligodendroctyomas.

### Administration

In one aspect of the present disclosure the OGFR antagonist is administered locally adjacent to the tumor site. In some embodiments, the OGFR antagonist is administered at a local dosage of about 1-mM to about 21-mM. In some embodiments, the OGFR antagonist is administered at a local dosage of about 1-mM to about 10-mM. In some embodiments, the local dosage is 1-mM, 1.5-mM, 2.0-mM, 3.0-mM, 4.0-mM, 5.0-mM, 6.0-mM, 7.0-mM, 8.0-mM, 9.0-mM, 10.0-mM, 11.0-mM, 12.0-mM, 13.0-mM, 14.0-mM, 15.0-mM, 16.0-mM, 17.0-mM, 18.0-mM, 19.0-mM, 20.0-mM, and/or 21.0-mM.

In some embodiments, the OGFR antagonist is administered with a carrier, and the carrier volume may be from about 0.1 cubic centimeter (cc) to about 20 cc, from about 0.25 cc to about 15 cc, from about 0.5 cc to about 10 cc, from about 1 cc to about 10 cc, from about 2 cc to about 10 cc. The corresponding amount of the OGFR antagonist in the carrier may be from about 0.2 mg per cc, 0.5 mg per cc, 1.0 mg per cc, 2.0 mg per cc, 3.0 mg per cc, 4.0 mg per cc, 5.0 mg per cc, 6.0 mg per cc, 7.0 mg per cc, 8.0 mg per cc, 9.0 mg per cc, and/or 10.0 mg per cc.

In some embodiments, the OGFR antagonist is administered locally by intratumoral injection.

In some embodiments, an OGFR antagonist is combined with or encapsulated within a carrier for administration.

Suitable carriers can be in bead, microsphere or nanoparticle form, and can be made of natural and/or synthetic biocompatible polymers. Examples of suitable biocompatible polymers include hyaluronic acid, collagen, tricalcium phosphate, chondroitin sulfate, polybutyrate, polylactide, polyglycolide, and lactide/glycolide copolymers, and mixtures or copolymers thereof. Suitable carriers also include on-polymer systems such as carboxylic acids, fatty acids, phospholipids, amino acids, lipids such as sterols, hydrogel release system; silastic system; peptide-based system; implants and the like.

In one embodiment, the carrier is a hygroscopic collagen based carrier such as a collagen sponge, a collagen scaffold, a powdered collagen, or a collagen based gelatin hydrogel.

In another embodiment, the carrier is a hydrophilic hydrogel based carrier (e.g., poly lactic acid, poly glycolic acid), which allows an OGFR antagonist (e.g., naloxone or naltrexone or a functional derivative thereof) infused therein to be released over a period of time.

In another embodiment, the carrier is a carrier composed of a tri-block co-polymer comprising a central block of PLA (poly-(lactic acid) flanked by two blocks of PEG-(poly-(ethylene glycol).

In still another embodiment, the carrier is albumin, a derivative or fragment of albumin that maintains the naloxone/morphine binding site located at the interface between the IA and IIA domains, and/or maintains the naloxone binding site around tryptophan (Trp)-214, that binds an OGFR antagonist such as naloxone or naltrexone or a functional derivative thereof and allows for a slow release of the OGFR antagonist. In still another embodiment, methyl cellulose, and an inert gel, for example, that binds an OGFR antagonist such as naloxone or naltrexone or a functional derivative thereof and allows for a slow release of the antagonist.

In a further embodiment, the carrier is a bovine collagen implant. An OGFR antagonist, e.g., naloxone or naltrexone or a functional derivative thereof, can be combined with a bovine collagen implant, that is supplied with a bovine collagen sponge, powdered bovine collagen, or collagen based gelatin construct. Administration of naloxone, naltrexone or a functional derivative thereof can be achieved by, e.g., reconstituting the powdered naloxone or naltrexone or a functional derivative thereof with sterile saline and then adding the OGFR antagonist-saline solution to the collagen implant; after which the implant can be delivered locally to the site of surgical intervention.

In another embodiment, the carrier is a carrier composed of PGA (poly-(glycolic acid)-PLGA (poly-(lactic glycolic acid)) spheres, which can encapsulate an OGFR antagonist to provide for immediate, delayed or sustained release.

### Pharmaceutical Compositions

In some embodiments, a pharmaceutical formulation may be a pharmaceutical disclosed further comprising one or more additional “pharmaceutically acceptable carriers,” such as an aqueous carrier, buffer, antioxidants, and/or diluents. In some embodiments, the pharmaceutical compositions comprise an OGFR antagonist in a dimethyl sulfoxide (DMSO) based saline solution. In some embodiments, the pharmaceutical compositions comprise an OGFR antagonist in an ethanol based saline solution.

In some embodiments, the acidified saline based solution exhibits a pH from about 4.5 to about 7.4. In some embodiments, the acidified saline based solution exhibits a pH from about 5.5 to about 7.4. In some embodiments, the acidified saline based solution exhibits a pH from about 6.5 to about 7.4.

In some embodiments, the diluent is a dimethyl sulfoxide (DMSO) based saline solution. In some embodiments, the DMSO based saline solution comprises from about 1% volume/volume (v/v) DMSO to about 80% v/v DMSO. In some embodiments, the DMSO based saline solution comprises from about 5% volume/volume (v/v) DMSO to about 80% v/v DMSO. In some embodiments, the DMSO based saline solution comprises from about 10% volume/volume (v/v) DMSO to about 80% v/v DMSO. In some embodiments, the DMSO based saline solution comprises from about 15% volume/volume (v/v) DMSO to about 80% v/v DMSO. In some embodiments, the DMSO based saline solution comprises from about 25% volume/volume (v/v) DMSO to about 80% v/v DMSO. In some embodiments, the DMSO based saline solution comprises from about 30% volume/volume (v/v) DMSO to about 80% v/v DMSO. In some embodiments, the DMSO based saline solution comprises from about 35% volume/volume (v/v) DMSO to about 80% v/v DMSO. In some embodiments, the DMSO based saline solution comprises from about 40% volume/volume (v/v) DMSO to about 80% v/v DMSO. In some embodiments, the DMSO based saline solution comprises from about 40% volume/volume (v/v) DMSO to about 70% v/v DMSO.

In some embodiments, the DMSO based saline solution comprises about 1% volume/volume (v/v) DMSO. In some embodiments, the DMSO based saline solution comprises about 2% volume/volume (v/v) DMSO. In some embodiments, the DMSO based saline solution comprises about 3% volume/volume (v/v) DMSO. In some embodiments, the DMSO based saline solution comprises about 4% volume/volume (v/v) DMSO. In some embodiments, the DMSO based saline solution comprises about 5% volume/volume (v/v) DMSO. In some embodiments, the DMSO based saline solution comprises about 6% volume/volume (v/v) DMSO. In some embodiments, the DMSO based saline solution comprises about 7% volume/volume (v/v) DMSO. In some embodiments, the DMSO based saline solution comprises about 8% volume/volume (v/v) DMSO. In some embodiments, the DMSO based saline solution comprises about 9% volume/volume (v/v) DMSO. In some embodiments, the DMSO based saline solution comprises about 10% volume/volume (v/v) DMSO. In some embodiments, the DMSO based saline solution comprises about 20% volume/volume (v/v) DMSO. In some embodiments, the DMSO based saline solution comprises about 30% volume/volume (v/v) DMSO. In some embodiments, the DMSO based saline solution comprises about 40% volume/volume (v/v) DMSO. In some embodiments, the DMSO based saline solution comprises about 50% volume/volume (v/v) DMSO. In some embodiments, the DMSO based saline solution comprises about 60% volume/volume (v/v) DMSO. In some embodiments, the DMSO based saline solution comprises about 70% volume/volume (v/v) DMSO. In some embodiments, the DMSO based saline solution comprises about 80% volume/volume (v/v) DMSO.

In some embodiments, the dimethyl sulfoxide (DMSO) based saline solution comprises a phosphate buffered saline solution, a borate buffered saline solution, a Tris buffered saline solution, or a carbonate buffered saline solution.

In some embodiments, the diluent is an ethanol based saline solution. In some embodiments, the ethanol based saline solution comprises from about 1% volume/volume (v/v) ethanol to about 80% v/v ethanol. In some embodiments, the ethanol based saline solution comprises from about 5% volume/volume (v/v) ethanol to about 80% v/v ethanol. In some embodiments, the ethanol based saline solution comprises from about 10% volume/volume (v/v) ethanol to about 80% v/v ethanol. In some embodiments, the ethanol based saline solution comprises from about 15% volume/volume (v/v) ethanol to about 80% v/v ethanol. In some embodiments, the ethanol based saline solution comprises from about 25% volume/volume (v/v) ethanol to about 80% v/v ethanol. In some embodiments, the ethanol based saline solution comprises from about 30% volume/volume (v/v) ethanol to about 80% v/v ethanol. In some embodiments, the ethanol based saline solution comprises from about 35% volume/volume (v/v) ethanol to about 80% v/v ethanol. In some embodiments, the ethanol based saline solution comprises from about 40% volume/volume (v/v) ethanol to about 80% v/v ethanol. In some embodiments, the ethanol based saline solution comprises from about 40% volume/volume (v/v) ethanol to about 70% v/v ethanol.

In some embodiments, the ethanol based saline solution comprises about 1% volume/volume (v/v) ethanol. In some embodiments, the ethanol based saline solution comprises about 2% volume/volume (v/v) ethanol. In some embodiments, the ethanol based saline solution comprises about 3% volume/volume (v/v) ethanol. In some embodiments, the ethanol based saline solution comprises about 4% volume/volume (v/v) ethanol. In some embodiments, the ethanol based saline solution comprises about 5% volume/volume (v/v) ethanol. In some embodiments, the ETHANOL based saline solution comprises about 6% volume/volume (v/v) ethanol. In some embodiments, the ethanol based saline solution comprises about 7% volume/volume (v/v) ethanol. In some embodiments, the ethanol based saline solution comprises about 8% volume/volume (v/v) ethanol. In some embodiments, the ethanol based saline solution comprises about 9% volume/volume (v/v) ethanol. In some embodiments, the ethanol based saline solution comprises about 10% volume/volume (v/v) ethanol. In some embodiments, the ethanol based saline solution comprises about 20% volume/volume (v/v) ethanol. In some embodiments, the ethanol based saline solution comprises about 30% volume/volume (v/v) ethanol. In some embodiments, the ethanol based saline solution comprises about 40% volume/volume (v/v) ethanol. In some embodiments, the ethanol based saline solution comprises about 50% volume/volume (v/v) ethanol. In some embodiments, the ethanol based saline solution comprises about 60% volume/volume (v/v) ethanol. In some embodiments, the ethanol based saline solution comprises about 70% volume/volume (v/v) ethanol. In some embodiments, the ethanol based saline solution comprises about 80% volume/volume (v/v) ethanol.

In some embodiments, the ethanol based saline solution comprises a phosphate buffered saline solution, a borate buffered saline solution, a Tris buffered saline solution, or a carbonate buffered saline solution.

In some embodiments, the saline solution comprises a salt and water. In some embodiments, the salt of the saline solution comprises sodium chloride, or potassium chloride. In some embodiments, the saline solution comprises from about 0.7% w/w salt to about 1.5% w/w salt. In some embodiments, the saline solution comprises about 0.7% salt. In some embodiments, the saline solution comprises about 0.8% salt. In some embodiments, the saline solution comprises about 0.9% salt. In some embodiments, the saline solution comprises about 1.0% salt. In some embodiments, the saline solution comprises about 1.1% salt. In some embodiments, the saline solution comprises about 1.2% salt. In some embodiments, the saline solution comprises about 1.3% salt. In some embodiments, the saline solution comprises about 1.4% salt. In some embodiments, the saline solution comprises about 1.5% salt.

The OGFR antagonist may be combined or coordinately administered with a suitable carrier or vehicle depending on the route of administration. The term “pharmaceutically acceptable carrier” refers to a carrier that is conventionally used in the art to facilitate the storage, administration, and/or the healing effect of an active agent of a pharmaceutical composition.

A water-containing liquid carrier can comprise pharmaceutically acceptable additives such as acidifying agents, alkalizing agents, antimicrobial preservatives, antioxidants, buffering agents, chelating agents, complexing agents, solubilizing agents, humectants, solvents, suspending and/or viscosity-increasing agents, tonicity agents, wetting agents or other biocompatible materials. A tabulation of ingredients listed by the above categories can be found in the U.S. Pharmacopeia National Formulary, 1857-1859, and (1990). Some examples of the materials which can serve as pharmaceutically acceptable carriers are sugars, such as lactose, glucose and sucrose; cyclodextrins, including alpha-cyclodextrin, beta-cyclodextrin, and gamma-cyclodextrin; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols, such as propylene glycol; polyols such as glycerin, sorbitol, mannitol and polyethylene glycol; esters such as ethyl oleate and ethyl laurate; agar; buffering agents such as magnesium hydroxide and aluminum hydroxide; alginic acid; pyrogen free water; isotonic saline; Ringer's solution, ethyl alcohol and phosphate buffer solutions, as well as other nontoxic compatible substances used in pharmaceutical formulations. Wetting agents, emulsifiers and lubricants such as sodium lauryl sulfate and magnesium stearate, as well as coloring agents, release agents, coating agents, sweetening, flavoring and perfuming agents.

### Calcium Phosphate Bone Graft Composition

A bone graft composition, or material, according to an embodiment facilitates repair or regeneration of bone at a target repair site. For example, in some embodiments, the bone graft composition can be osteoconductive, osteoinductive, bioactive, osteostimulative, antibacterial or any combination thereof. The target repair site can be, for example, a void, gap, or other defect in a bone or other bony structure in a body of a patient. For example, as described in more detail below, the bone graft composition facilitates bone growth at a target repair site in the spine, pelvis, an extremity, the cranium, or another bone or bony structure in the patient's body. The bone graft composition can be implanted, extruded, molded, or otherwise placed at the target repair site. For example, in some embodiments, the bone graft composition can be implanted, extruded, molded, or placed at the target repair site in a non-load bearing application. In other embodiments, the bone graft composition can be implanted, extruded, molded, or placed at the target repair site with appropriate orthopedic ‘hardware’ (i.e. screws, plates, rods, cages/spacers, prosthetics, hip implants, knee implants, acetabular implants, etc.) in load bearing applications.

In some embodiments, the bone graft composition is a calcium phosphate composition.

In some embodiments, the calcium phosphate is chosen from the following: hydroxyapatite (Ca₅(OH)(PO₄)₃), beta-tricalcium phosphate (beta-Ca₃(PO₄)₂), calcium phosphate dibasic (CaHPO₄), or calcium phosphate tribasic (Ca₅(OH)(PO₄)₃), monocalcium phosphate monohydrate (Ca(H₂PO₄)₂·H₂O), dicalcium phosphate dihydrate (CaHPO₄·₂H₂O), octocalcium phosphate (Ca₈H₂(PO₄)₆·₅H₂O), monocalcium phosphate (Ca(H₂PO₄)₂), alpha-tricalcium phosphate (alpha-Ca₃(PO₄)₂), sintered hydroxyapatite (Ca₁₀(PO₄)₆(OH)₂), oxyapatite (Ca₁₀(PO₄)₆O), or tetracalcium phosphate (Ca₄(PO₄)₂O).

In some embodiments, the hydroxyapatite is a precipitated hydroxyapatite material with the chemical formula Ca₁₀₋ₓ (HPO₄)ₓ(PO₄)₆₋ₓ (OH)₂₋ₓ, in which ‘x’ may vary between 0 and 2.

In some embodiments, the tricalcium phosphate is a precipitated amorphous calcium phosphate with the chemical formula Ca₃(PO₄)₂·nH₂O, in which ‘n’=3-4.5 and the H2O content is 15%-20%.

In some embodiments, the size of individual grains (or particles) of the hydroxyapatite material ranges from 1-nm to 5-mm.

In some preferred embodiments, the grain size for the hydroxyapatite will be selected from the following sizes: 10-micron, 75-micron, 86.4-micron, 125-micron, 147-micron, 212-micron, 368-micron, or 740-micron. In these embodiments, the grain size will be understood to represent the max size and that a range will exist with potentially smaller particles of the material.

In some embodiments, the size of individual grains (or particles) of the beta-tricalcium phosphate material ranges from 1-nm to 5-mm.

In some preferred embodiments, the grain size of the beta-tricalcium phosphate will be selected from the following sizes: 100-micron, 125-micron, 212-micron, 304-micron, 645-micron, 500-micron, or 1000-micron. In these embodiments, the grain size will be understood to represent the max size and that a range will exist with potentially smaller particles of the material.

In some embodiments, the tricalcium phosphate will be a mixture of beta-tricalcium phosphate and alpha-tricalcium phosphate.

In some embodiments, the calcium phosphate salts could be anhydrous, monohydrate, dihydrate, or any xH₂O hydrate isoform.

In a preferred embodiment, the calcium phosphate salts contained in the preferred formulation of the bone graft material are sintered hydroxyapatite (Ca₁₀(PO₄)₆(OH)₂), beta-tricalcium phosphate (beta-Ca₃(PO₄)₂), calcium phosphate dibasic (CaHPO₄), and calcium phosphate tribasic (Ca₅(OH)(PO₄)₃).

### Hardening Agents

In some embodiments, the bone graft composition comprises a hardening agent. A hardening agent improves the degree of hardening of the bone graft composition.

In some embodiments, the hardening agent may be sodium carbonate (Na₂CO₃).

In some embodiments, the hardening agent may also be calcium sulfate anhydrate (CaSO₄), calcium sulfate hemihydrate (CaSO₄·0.5H₂O), calcium sulfate dihydrate (CaSO₄·₂H₂O), calcium carbonate (CaCO₃), magnesium carbonate (MgCO₃), strontium carbonate (SrCO₃), and bioglass.

In some embodiments, the hardening agent salts could be anhydrous, monohydrate, dihydrate, or any xH₂O hydrate isoform.

In a preferred embodiment, the hardening agent contained in the preferred formulation of the bone graft material is sodium carbonate (Na₂CO₃).

Agents that Control the Rate of Curing

In some embodiments, the bone graft composition comprises a curing agent. A curing agent improves the rate of curing of the bone graft composition.

In some embodiments, the curing agent may comprise calcium oxide (CaO), magnesium oxide (MgO), sodium phosphate dibasic (Na₂HPO₄), sodium pyrophosphate tetrabasic (Na₄P₂O₇), sodium orthophosphate (Na₃PO₄), or sodium phosphate monobasic (NaH₂PO₄).

In some embodiments, the sodium phosphate salts may comprise anhydrous, monohydrate, dihydrate, or any xH₂O hydrate isoform.

In a preferred embodiment, the agents that control the rate of curing comprise calcium oxide (CaO), magnesium oxide (MgO), and sodium phosphate dibasic (Na₂HPO₄).

### Acidifying Agents

It is a discovery of the present inventors that adding acidifying agents to a bone graft composition provides improved material properties, dissolution, bioavailability of small molecules, promotes bone formation, and improves handling properties. An acidifying agent is defined as a chemical agent, molecule, or ion capable of donating a proton (hydrogen ion H+; known as a Brønsted-Lowry acids); or, alternatively, capable of forming a covalent bond with an electron pair (known as a Lewis acid).

In some embodiments, the bone graft composition comprises the acidifying agent, wherein the acidifying agent comprises L-ascorbic acid (C₆H₈O₆), ascorbate 2-phosphate sesquimagnesium salt hydrate (C₁₂H₁₂O₁₀(PO₄)₂Mg₃·xH₂O), sodium citrate dihydrate (Na₃C₆H₅O₇·₂H₂O), magnesium citrate (MgC₆H₆O₇), potassium citrate monohydrate (K₃C₆H₅O₇·H₂O), citric acid monohydrate (C₆H₈O₇·H₂O), and acetic acid (C₂H₄O₂).

In some embodiments, the acidifying agent could be anhydrous, monohydrate, dihydrate, or any xH₂O hydrate isoform.

In some embodiments, the acidifying agent is a component of the bone graft material and is a salt or ester chemical species that produces a polyatomic anion that forms in a solution and when in solution otherwise conforms to the conventions that define a chemical species as an acid.

In a preferred embodiment, the acidifying agent comprises one or more of L-ascorbic acid (C₆H₈O₆), citric acid monohydrate (C₆H₈O₇·H₂O), and acetic acid (C₂H₄O₂).

### Iron Excipients

It is a discovery of the present inventors that adding iron excipients, in the form of iron salts, iron oxides, iron compounds, iron alloys, and elemental iron to the bone graft composition enables the following: 1) As a chromogenic agent, iron excipients provide for easy detection of where the putty is relative to the adjacent bone. In addition, iron excipients as chromogenic agents allow visualization of adequate mixing based on evaluating the uniformity of color upon mixing the liquid and solid precursors of the bone graft composition. 2) Iron excipients modulate the material properties of the bone graft material by functioning as agents that control the rate of curing and also as agents that contribute to hardening. 3) Iron excipients act as contrast agents during x-radiographic imaging (X-radiographs and computed tomography). 4) Iron excipients contribute to osteogenesis.

In some embodiments, chromogenic agents comprise a chromogen that is also an iron excipient. As used herein, the term “chromogen” is any chemical species that is an iron excipient that also changes color upon dissolving in a solvent.

In some embodiments, chromogenic agents comprise an activateable chromogenic agent.

In some embodiments, the chromogenic agent colors the bone graft composition red, orange, brown, black, or grey. In some preferred embodiments, the chromogenic agent colors the bone graft composition orange, red, brown, or grey.

In some embodiments, the iron excipient is iron (III) sulfate hydrate (Fe₂(SO₄)₃), iron (III) chloride (FeCl₃), iron (III) citrate (FeC₆H₅O₇), iron (III) oxide (Fe₂O₃), iron (III) hydroxide oxide (Fe(OH)O), iron (III) phosphate (FePO₄), ammonium iron (III) citrate (C₆H₈O₇·xFe₃+yNH₃), elemental iron particles (Fe), iron (III) fluoride (FeF₃), iron (II) sulfate heptahydrate (FeSO₄·₇H₂O), iron (II) ammonium sulfate hexahydrate (Fe(NH₄)₂(SO₄)₂·₆H₂O), iron (II) chloride (FeC₁₂), iron (II) disulfide (FeS₂), iron (II) sulfate heptahydrate (FeSO₄·₇H₂O), and iron (II) lactate hydrate (Fe(CH₃CH(OH)COO)₂·xH₂O), iron (II) L-ascorbate (FeC₁₂H₁₄O₁₂), and iron (II) fluoride (FeF₂).

In a preferred embodiment, the iron excipient is iron (III) sulfate hydrate (Fe₂(SO₄)₃).

### Collagen

It is a discovery of the present inventors that collagen powders and liquid collagen preparations can be used to create a bone graft material. Type-I collagen is a white, hygroscopic material commonly used as a component of bone grafts.

Collagen powders are preparations of a collagen material in which the collagen polymer is cleaved chemically or enzymatically or through any conventional means that produces fragments of collagen of a particular size or range of sizes. In this preparation, the collagen could be 1-nm to 100-micron produced through reverse dialysis. In another preparation, the collagen could be cleaved enzymatically to produce fragments between 100-micron to 300-micron. In yet another preparation, the collagen could be ground or milled mechanically and then passed through a filter to produce fragments between 200-micron to 5-mm. In another embodiment, the collagen has a particle size of from 25-microns to 750-microns. In another embodiment, the collagen has a particle size of from 1-micron to 1.5-mm. In still yet another preparation, collagen is ground or milled at low temperatures (cryogrinding) to produce fragments from 1-micron to 5-mm.

A liquid collagen is a preparation of collagen that contains the soluble fraction of collagen that is dissolved in a solution. In this preparation, the collagen is soaked in an acid solution, and/or it is heated between 100-degrees Celsius and 300-degrees Celsius, and/or it is heated in a pressurized environment, and/or it is filtered to produce a fragment of a particular size. In yet another preparation, the liquid collagen is produced through grinding or milling collagen suspended in a solution using rotor stator to produce collagen fragments between 1-micron to 1-mm.

In a preferred preparation, the collagen is milled to produce powder with one of the following sized fragments: 10-micron, 15-micron, 20-micron, 25-micron, 30-micron, 35-micron, 40-micron, 50-micron, 55-micron, 60-micron, 65-micron, 70-micron, 75-micron, 80-micron, 85-micron, 90-micron, 95-micron, 100-micron, 150-micron, 200-micron, 250-micron, 300-micron, 350-micron, 400-micron, 450-micron, 500-micron, 750-micron, or 1000-micron fragment. In another preparation, the collagen powder is composed of a range of fragment sizes from the above.

### Diluent Solution

A diluent solution is an aqueous media composed of water and other excipients that is able to make soluble the powder or dry slurry component of a bone graft composite material.

In some embodiments, the bone graft composition comprises a diluent or a solution that is added to the other components of the bone graft material in order to produce a malleable, moldable putty.

In some embodiments, the diluent is saline or water.

In a preferred embodiment, the diluent is a 0.9% saline solution as is commonly used in several medical and scientific applications.

In another preferred embodiment, the diluent is a 0.9% saline solution that contains acidifying agents.

In yet another preferred embodiment, the diluent is a 0.9% saline solution that contains acetic acid.

In still yet another preferred embodiment, the diluent is a 0.9% saline solution that contains acetic acid and any of the acidifying agents that contain a citric acid molecule, such as citric acid monohydrate or magnesium citrate, potassium citrate, or sodium citrate dihydrate.

In another preferred embodiment, the diluent is a 0.9% saline solution that contains acidifying agents and a liquid preparation of collagen.

### Preparation of Bone Graft Compositions

As used herein, the term “blending” refers to the physical process that combines or puts together non-aqueous materials to form one substance or mass that is a powder, or a dry slurry referred to herein as a “powder-dry slurry”. Part of preparing a bone graft composite material requires the blending of the following constituents into a single powder-dry slurry composite material: calcium phosphates with hardening agents, iron excipients, agents that control the rate of curing, acidifying agents, and powdered collagen.

As used herein, the term “mixing” refers to a physical process that combines a powder-dry slurry with a diluent solution to form a paste, putty, or otherwise amorphous solid.

In some embodiments, the pharmaceutical composition comprises preservatives and antioxidants. Examples of pharmaceutically acceptable antioxidants include water soluble antioxidants such as ascorbic acid, cysteine hydrochloride, sodium bisulfite, sodium metabisulfite, sodium sulfite and the like; oil-soluble antioxidants such as ascorbyl palmitate, butylated hydroxyanisole (BHA), butylated hydroxytoluene (BHT), lecithin, propyl gallate, alpha-tocopherol and the like; and metal-chelating agents such as citric acid, ethylenediamine tetraacetic acid (EDTA), sorbitol, tartaric acid, phosphoric acid and the like. Pharmaceutical compositions according to the invention may also comprise one or more binding agents, filling agents, lubricating agents, suspending agents, sweeteners, flavoring agents, preservatives, buffers, wetting agents, disintegrants, effervescent agents, and other excipients. Such excipients are known in the art. Examples of filling agents include lactose monohydrate, lactose anhydrous, and various starches; examples of binding agents include various celluloses and cross-linked polyvinylpyrrolidone, microcrystalline cellulose such as Avicel™, PH101 microcrystalline cellulose and/or Avicel™, PH102 microcrystalline cellulose, and silicified microcrystalline cellulose such as ProSolv SMCC™. Suitable lubricants, including agents that act on the flow-ability of the powder to be compressed, may include colloidal silicon dioxide such as Aerosil® 200 (colloidal silicon dioxide), talc, stearic acid, magnesium stearate, calcium stearate, and silica gel. Examples of sweeteners may include any natural or artificial sweetener, such as sucrose, xylitol, sodium saccharin, cyclamate, aspartame, and acesulfame. Examples of flavoring agents are Monoammonium Glycyrrhizinate such as Magnasweet™ (a flavoring composition containing Monoammonium Glycyrrhizinate and trademark of MAFCO), bubble gum flavor, and fruit flavors, and the like. Examples of preservatives include potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quaternary compounds such as benzalkonium chloride.

Any pharmaceutical used for therapeutic administration can be sterile. Sterility is readily accomplished by for example filtration through sterile filtration membranes (e.g., 0.2 micron membranes). Any pharmaceutically acceptable sterility method can be used in the compositions of the invention. The pharmaceutical composition comprising an OGFR antagonist derivatives or salts thereof will be formulated and dosed in a fashion consistent with good medical practice, taking into account the clinical condition of the individual patient, the method of administration, the scheduling of administration, and other factors known to practitioners.

A variety of administration routes are available. The pharmaceutical composition of the invention may be practiced using any mode of administration that is medically acceptable, meaning any mode that produces effective levels of the active ingredients without causing clinically unacceptable adverse effects. Accordingly, the pharmaceutical compositions can be administered to a subject parenterally, orally, intraperitoneally, intravenously, intra-arterially, transdermally, sublingually, intramuscularly, intratumorally, intraarticularly, rectally, transbuccally, intranasally, liposomally, via minicells, via antibody conjugation, via cell targeting peptides, via inhalation, vaginally, intraocularly, via local delivery by catheter or stent, subcutaneously, intra-adiposally, intra-articularly, or intrathecally. Modes of administration include oral, rectal, topical, nasal, intradermal, or parenteral routes, with the term “parenteral” being understood to include subcutaneous, intravenous, intramuscular, or infusion.

Surgical administration—in other words, the means of delivering the pharmaceutical composition via a surgical intervention, which includes open procedures, minimally invasive procedures, hybrid minimally invasive open procedures, image guided surgical interventions via fluoroscopy or ultrasound, and laparoscopic interventions. Surgical interventions can be in any soft tissue or in any bone tissues, including cortical bone, trabecular bone, bone marrow, spinal bones, pelvic bones, the axial skeleton, the appendicular skeleton, and the cranium.

Preparations for administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's solution or fixed 25 oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Lower doses will result from other forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of compounds.

Other delivery systems can include time-release, delayed-release, or sustained-release delivery systems. Such systems can avoid repeated administrations of the pharmaceutical composition of this invention, increasing convenience to the subject and the physician. Many types of release delivery systems are available and known to those of ordinary skill in the art. They include polymer-based systems such as poly (lactide-glycolide), copolyoxalates, polycaprolactones, polyesteramides, polyorthoesters, polyhydroxybutyric acid, and polyanhydrides. Microcapsules of the foregoing polymers containing drugs are described in U.S. Pat. No. 5,075,109, for example. Delivery systems also include non-polymer systems that are: lipids, including sterols such as cholesterol, cholesterol esters, and fatty acids or neutral fats such as mono-, di-, and tri-glycerides; hydrogel release systems; sylastic systems; peptide-based systems; wax coatings; compressed tablets using conventional binders and excipients; partially fused implants; and the like.

### Cancer

In some embodiments, the controlled release formulation may be used for treating a cancer.

Cancer may refer to a condition in which abnormal cells divide without control and can invade nearby tissues.

In some embodiments, the cancer may be a prostate cancer or a cancer that derives from a prostate cancer, e.g., metastases.

The methods and compositions herein may be provided in the form of a kit. A “kit” is herein defined as a package and containing several individual parts that show a complementary effect when applied together. In this aspect, the effect achieved by a kit and the pharmaceutical composition are similar. The kit may optionally include instructions for using the pharmaceutical compositions.

The present invention is further illustrated by, though in no way limited to, the following examples.

The following design parameters were used to determine a design space in which ≤100% of the drug release occurred over 24-hours, and the material could be implanted or injected using a cannula. A summary of the compositions that met these design criteria are listed below in Table I.

TABLE I
Combinations of Components for the Preferred Formulation

Component

| | Percentage of Component (w/w) | | | | | | | | | |
|---|---|---|---|---|---|---|---|---|---|---|
| Iron(III) sulfate | 5.0% | 5.1% | 5.2% | 5.3% | 5.4% | 5.5% | 5.6% | 5.7% | 5.8% | 5.9% |
| | 6.0% | 6.1% | 6.2% | 6.3% | 6.4% | 6.5% | 6.6% | 6.7% | 6.8% | 6.9% |
| | 7.0% | 7.1% | 7.2% | 7.3% | 7.4% | 7.5% | 7.6% | 7.7% | 7.8% | 7.9% |
| | 8.0% | 8.1% | 8.2% | 8.3% | 8.4% | 8.5% | 8.6% | 8.7% | 8.8% | 8.9% |
| | 9.0% | 9.1% | 9.2% | 9.3% | 9.4% | 9.5% | 9.6% | 9.7% | 9.8% | 9.9% |
| | 10.0% | 10.1% | 10.2% | 10.3% | 10.4% | 10.5% | 10.6% | 10.7% | 10.8% | 10.9% |
| | 11.0% | 11.1% | 11.2% | 11.3% | 11.4% | 11.5% | 11.6% | 11.7% | 11.8% | 11.9% |
| | 12.0% | | | | | | | | | |
| calcium | 8.0% | 8.1% | 8.2% | 8.3% | 8.4% | 8.5% | 8.6% | 8.7% | 8.8% | 8.9% |
| phosphate | 9.0% | 9.1% | 9.2% | 9.3% | 9.4% | 9.5% | 9.6% | 9.7% | 9.8% | 9.9% |
| dibasic | 10.0% | 10.1% | 10.2% | 10.3% | 10.4% | 10.5% | 10.6% | 10.7% | 10.8% | 10.9% |
| | 11.0% | | | | | | | | | |
| calcium | 9.0% | 9.1% | 9.2% | 9.3% | 9.4% | 9.5% | 9.6% | 9.7% | 9.8% | 9.9% |
| phosphate | 10.0% | 10.1% | 10.2% | 10.3% | 10.4% | 10.5% | 10.6% | 10.7% | 10.8% | 10.9% |
| tribasic | 11.0% | 11.1% | 11.2% | 11.3% | 11.4% | 11.5% | 11.6% | 11.7% | 11.8% | 11.9% |
| | 12.0% | 12.1% | 12.2% | 12.3% | 12.4% | 12.5% | | | | |
| hydroxyapatite | 13.0% | 13.1% | 13.2% | 13.3% | 13.4% | 13.5% | 13.6% | 13.7% | 13.8% | 13.9% |
| | 14.0% | 14.1% | 14.2% | 14.3% | 14.4% | 14.5% | 14.6% | 14.7% | 14.8% | 14.9% |
| | 15.0% | 15.1% | 15.2% | 15.3% | 15.4% | 15.5% | 15.6% | 15.7% | 15.8% | 15.9% |
| | 16.0% | 16.1% | 16.2% | 16.3% | 16.4% | 16.5% | 16.6% | 16.7% | 16.8% | 16.9% |
| | 17.0% | | | | | | | | | |
| beta-tricalcium | 3.0% | 3.1% | 3.2% | 3.3% | 3.4% | 3.5% | 3.6% | 3.7% | 3.8% | 3.9% |
| phosphate | 4.0% | 4.1% | 4.2% | 4.3% | 4.4% | 4.5% | 4.6% | 4.7% | 4.8% | 4.9% |
| | 5.0% | 5.1% | 5.2% | 5.3% | 5.4% | 5.5% | 5.6% | 5.7% | 5.8% | 5.9% |
| | 6.0% | | | | | | | | | |
| sodium | 4.0% | 4.1% | 4.2% | 4.3% | 4.4% | 4.5% | 4.6% | 4.7% | 4.8% | 4.9% |
| phosphate | 5.0% | 5.1% | 5.2% | 5.3% | 5.4% | 5.5% | 5.6% | 5.7% | 5.8% | 5.9% |
| dibasic | 6.0% | 6.1% | 6.2% | 6.3% | 6.4% | 6.5% | 6.6% | 6.7% | 6.8% | 6.9% |
| | 7.0% | 7.1% | 7.2% | 7.3% | 7.4% | 7.5% | | | | |
| sodium | 11.0% | 11.1% | 11.2% | 11.3% | 11.4% | 11.5% | 11.6% | 11.7% | 11.8% | 11.9% |
| carbonate | 12.0% | 12.1% | 12.2% | 12.3% | 12.4% | 12.5% | 12.6% | 12.7% | 12.8% | 12.9% |
| | 13.0% | 13.1% | 13.2% | 13.3% | 13.4% | 13.5% | 13.6% | 13.7% | 13.8% | 13.9% |
| | 14.0% | 14.1% | 14.2% | 14.3% | 14.4% | 14.5% | 14.6% | 14.7% | 14.8% | 14.9% |
| | 15.0% | | | | | | | | | |
| calcium oxide | 2.0% | 2.1% | 2.2% | 2.3% | 2.4% | 2.5% | 2.6% | 2.7% | 2.8% | 2.9% |
| | 3.0% | 3.1% | 3.2% | 3.3% | 3.4% | 3.5% | 3.6% | 3.7% | 3.8% | 3.9% |
| | 4.0% | 4.1% | 4.2% | 4.3% | 4.4% | 4.5% | 4.6% | 4.7% | 4.8% | 4.9% |
| | 5.0% | 5.1% | 5.2% | 5.3% | 5.4% | 5.5% | 5.6% | 5.7% | 5.8% | 5.9% |
| | 6.0% | | | | | | | | | |
| magnesium oxide | 0.5% | 0.6% | 0.7% | 0.8% | 0.9% | 1.0% | 1.1% | 1.2% | 1.3% | 1.4% |
| | 1.5% | 1.6% | 1.7% | 1.8% | 1.9% | 2.0% | 2.1% | 2.2% | 2.3% | 2.4% |
| | 2.5% | | | | | | | | | |
| Type-I Collagen | 18.0% | 18.1% | 18.2% | 18.3% | 18.4% | 18.5% | 18.6% | 18.7% | 18.8% | 18.9% |
| | 19.0% | 19.1% | 19.2% | 19.3% | 19.4% | 19.5% | 19.6% | 19.7% | 19.8% | 19.9% |
| | 20.0% | 20.1% | 20.2% | 20.3% | 20.4% | 20.5% | 20.6% | 20.7% | 20.8% | 20.9% |
| | 21.0% | 21.1% | 21.2% | 21.3% | 21.4% | 21.5% | 21.6% | 21.7% | 21.8% | 21.9% |
| | 22.0% | 22.1% | 22.2% | 22.3% | 22.4% | 22.5% | 22.6% | 22.7% | 22.8% | 22.9% |
| | 23.0% | | | | | | | | | |
| | Component Concentration (mM) | | | | | | | | | |
| naloxone | 1.00 | 1.10 | 1.20 | 1.30 | 1.40 | 1.50 | 1.60 | 1.70 | 1.80 | 1.90 |
| hydrochloride | 2.00 | 2.10 | 2.20 | 2.30 | 2.40 | 2.50 | 2.60 | 2.70 | 2.80 | 2.90 |
| | 3.00 | 3.10 | 3.20 | 3.30 | 3.40 | 3.50 | 3.60 | 3.70 | 3.80 | 3.90 |
| | 4.00 | 4.10 | 4.20 | 4.30 | 4.40 | 4.50 | 4.60 | 4.70 | 4.80 | 4.90 |
| | 5.00 | 5.10 | 5.20 | 5.30 | 5.40 | 5.50 | 5.60 | 5.70 | 5.80 | 5.90 |
| | 6.00 | 6.10 | 6.20 | 6.30 | 6.40 | 6.50 | 6.60 | 6.70 | 6.80 | 6.90 |
| | 7.00 | 7.10 | 7.20 | 7.30 | 7.40 | 7.50 | 7.60 | 7.70 | 7.80 | 7.90 |
| | 8.00 | 8.10 | 8.20 | 8.30 | 8.40 | 8.50 | 8.60 | 8.70 | 8.80 | 8.90 |
| | 9.00 | 9.10 | 9.20 | 9.30 | 9.40 | 9.50 | 9.60 | 9.70 | 9.80 | 9.90 |
| | 10.00 | 10.10 | 10.20 | 10.30 | 10.40 | 10.50 | 10.60 | 10.70 | 10.80 | 10.90 |
| | 11.00 | 11.10 | 11.20 | 11.30 | 11.40 | 11.50 | 11.60 | 11.70 | 11.80 | 11.90 |
| | 12.00 | 12.10 | 12.20 | 12.30 | 12.40 | 12.50 | 12.60 | 12.70 | 12.80 | 12.90 |
| | 13.00 | 13.10 | 13.20 | 13.30 | 13.40 | 13.50 | 13.60 | 13.70 | 13.80 | 13.90 |
| | 14.00 | 14.10 | 14.20 | 14.30 | 14.40 | 14.50 | 14.60 | 14.70 | 14.80 | 14.90 |
| | 15.00 | 15.10 | 15.20 | 15.30 | 15.40 | 15.50 | 15.60 | 15.70 | 15.80 | 15.90 |
| | 16.00 | 16.10 | 16.20 | 16.30 | 16.40 | 16.50 | 16.60 | 16.70 | 16.80 | 16.90 |
| | 17.00 | 17.10 | 17.20 | 17.30 | 17.40 | 17.50 | 17.60 | 17.70 | 17.80 | 17.90 |
| | 18.00 | 18.10 | 18.20 | 18.30 | 18.40 | 18.50 | 18.60 | 18.70 | 18.80 | 18.90 |
| | 19.00 | 19.10 | 19.20 | 19.30 | 19.40 | 19.50 | 19.60 | 19.70 | 19.80 | 19.90 |
| | 20.00 | 20.10 | 20.20 | 20.30 | 20.40 | 20.50 | 20.60 | 20.70 | 20.80 | 20.90 |
| | 21.00 | | | | | | | | | |

### EXAMPLES

The preferred embodiments contain the following as a percentage of weight: 1) between 5% and 12% of iron (III) sulfate, 2) between 8% and 11% calcium phosphate dibasic, 3) between 9% and 12.5% calcium phosphate tribasic, 4) between 13% and 17% hydroxyapatite, 5) between 3% and 6% beta-tricalcium phosphate, 6) between 4% and 7.5% sodium phosphate dibasic, 7) between 11% and 15% sodium carbonate, 8) between 2% and 6% calcium oxide, 9) between 4% and 6% magnesium oxide, 10) between 0.5% and 2.5% L-ascorbic acid, and 11) between 18% and 23% powdered type-I collagen. The preferred embodiment of the diluent component will be a solution between 0.5-mL and 12.5-mL in volume composed of the following: 1) a 0.9% saline solution, 2) between 2% and 6% of acetic acid, 3) between 0.1% and 2% citric acid monohydrate, and 4) between 0.01% and 2% of naloxone hydrochloride. Upon mixing the dry slurry component and the diluent, the bone graft material becomes moldable after approximately 2.5-minutes, turns a dark brown-color upon setting, and achieves a peak temperature of 38-degrees Celsius after 1.5-minutes of mixing.

### Example 1

Administration of the following specific example of the preferred embodiment was done with the following composition, identified as ZP001. ZP001, with the following composition, was added to cultures to test for anti-cancer activity: 1) 11.2% (w/w) iron (III) sulfate, 2) 8.7% (w/w) calcium phosphate dibasic, 3) 10.7% (w/w) calcium phosphate tribasic, 4) 15.2% (w/w) hydroxyapatite, 5) 4.5% (w/w) beta-tricalcium phosphate, 6) 5.8% (w/w) sodium phosphate dibasic, 7) 13.5% (w/w) sodium carbonate, 8) 4.5% (w/w) calcium oxide, 9) 4.5% (w/w) magnesium oxide, 10) 1.1% (w/w) L-ascorbic acid, 11) 20.2% powdered type-I collagen, 12) 0.9% saline solution, 13) 5% (v/v) acetic acid, 14) 0.9% (wt) citric acid monohydrate, and 4) 4-mg/mL of naloxone hydrochloride.

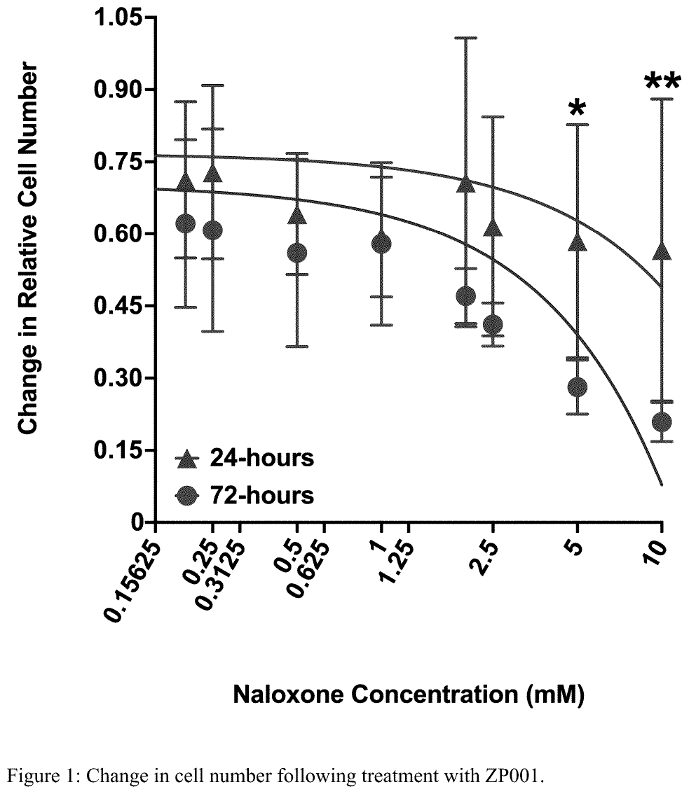

This formulation, ZP001, caused a significant decrease in prostate cancer cell (PC3 cells) number through 72-hours (FIG. 1) via cell death. These results were obtained as follows: The bone graft material was mixed as described and placed into cultures to assess the effects of ZP001 on prostate cancer tumor cells (PC3). Viable cell number was determined with the methyl tetrazolium (MTT) assay. After 72-hours of treatment, 5-mg/mL of the MTT reagent (w/v, Sigma) was added to each well and incubated for 2-hours, after which the cells were lysed with 500-μL of DMSO (Sigma).

50-μL of solution was added in duplicate to a 96-well plate and MTT absorbance was measured at 570-nm on a TECAN Spark monochromator. The effects on cell proliferation were determined by normalizing treated wells relative to mean values from non-treated wells, as follows: Fold change in cell number=100*[treated cells optical density/mean control optical density].

Culture studies demonstrated that the small molecule ZP001 reduced tumor cell number in a dose-dependent manner 72-hours after administration for the PC3 prostate cancer cells (FIG. 1). An Extra-Sum-of-Squares F-test demonstrated that the regression lines computed for PC3 cells at 24- and 72-hours were statistically different (p<0.0001). Specifically, ZP001 demonstrated a significant decrease of 171% at 72-hours versus at 24-hours (FIG. 1, *=p<0.0007). Correspondingly, treatment with ZP001 increased cell death in PC3 prostate cancer cells via the LDH assay through 120-hours (FIG. 2).

Tumor cell survival was also assessed using the spheroid assay, in which PC3 prostate cancer cells were grown and then treated with ZP001. Two weeks after administration of ZP001, the size of the spheroids had decreased significantly (FIG. 3).

Mouse Xenograft Studies: The animals in these studies were not treated with systemic chemotherapy, which meant there was no control of tumor dissemination beyond the local treatment area. This study was designed to assess whether ZP001 was non-tumorigenic. Male, 8-week-old, NCr nude athymic mice (N=24) were inoculated with 2×10⁵ PC3 prostate cancer cells into the bone marrow space of the tibia (day 0). Two weeks (14-days) after tumor inoculation, half of the animals (N=12) were implanted with a control implant (Control Implant) composed calcium phosphate based bone cement infused with 0.9% normal saline. The treatment group animals (N=12) were implanted with the ZP001. Implants were re-constituted immediately prior to implantation. Animals were monitored for an additional 23-days after implantation, during which time tumor volume was assessed with calipers 3-times every week. Animals with a tumor mass of ≥1000-mm3, signs of lameness, other pathologic signs of disease, and/or distress were euthanized.

Data were analyzed using a Malthusian exponential growth model using non-linear least-squares regression to estimate the doubling time for the tumor volume, which served as a surrogate for local tumor growth. Regression lines were assessed for statistical differences between groups using an Extra-Sum-of-Squares F-test to determine if the groups were statistically different or, alternatively, could be modeled using a single regression line (α=0.05).

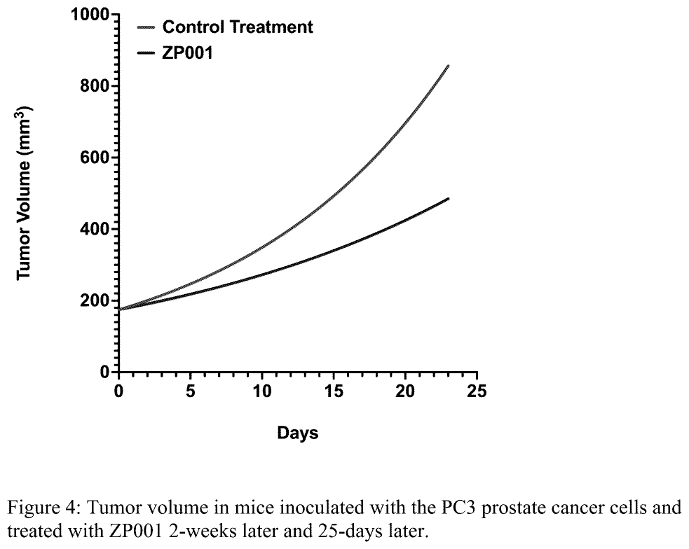

A Malthusian exponential growth model was used to estimate the rate of change in the tumor defect volume between the two treatment groups. The ZP001 treatment group was found to be significantly different from the Control Implant group using an Extra-Sum-of-Squares F-test, which demonstrated that the regression lines computed for PC3 tumors were statistically different (p<0.0001) (FIG. 4). Relative to the defect size at 14-days (day 0), the defect volume in the Control Implant group mice had increased 4.9-fold (FIG. 4). In contrast, treatment with ZP001 produced 2.8-fold increase in the tumor defect size in the tibia, which is almost a 2-fold decrease in tumor volume. Further, the Malthusian model predicted that the tumor doubling time had decreased 55.7% for the ZP001 treated animals versus the Control Implant group.

### Example 2

Administration of the following specific example of the preferred embodiment is performed with the following composition, identified as ZP002. ZP002, with the following composition, is added to cultures to test for anti-cancer activity: 1) 11.2% (w/w) iron (III) sulfate, 2) 8.7% (w/w) calcium phosphate dibasic, 3) 10.7% (w/w) calcium phosphate tribasic, 4) 15.2% (w/w) hydroxyapatite, 5) 4.5% (w/w) beta-tricalcium phosphate, 6) 5.8% (w/w) sodium phosphate dibasic, 7) 13.5% (w/w) sodium carbonate, 8) 4.5% (w/w) calcium oxide, 9) 4.5% (w/w) magnesium oxide, 10) 1.1% (w/w) L-ascorbic acid, 11) 20.2% powdered type-I collagen, 12) 0.9% saline solution, 13) 5% (v/v) acetic acid, 14) 0.9% (wt) citric acid monohydrate, and 4)₂-mg/mL of naloxone hydrochloride.

Although the foregoing refers to particular preferred embodiments, it will be understood that the present invention is not so limited. It will occur to those of ordinary skill in the art that various modifications may be made to the disclosed embodiments and that such modifications are intended to be within the scope of the present invention.

All of the publications, patent applications and patents cited in this specification are incorporated herein by reference in their entirety.

1.-7. (canceled)

8. A method of decreasing volume of a prostate cancer tumor comprising locally administering by introduction into the prostate cancer tumor of a subject in need thereof with a single injection a therapeutically effective amount of a composition comprising from about 3.2 mg/ml to about 5.2 mg/ml of naloxone or a salt thereof, iron (III) sulfate, calcium phosphate, and collagen, wherein the administration of the composition in a single injection decreases the volume of the prostate cancer tumor by about two-fold after 14 days compared to a phosphate-based bone cement infused with 0.9% normal saline.

9. (canceled)

10. (canceled)

11. The method of claim 8, wherein the subject is a human being.

12. (canceled)

13. The method of claim 8, wherein the composition further comprises hydroxyapatite, sodium carbonate, calcium oxide, magnesium oxide, L-ascorbic acid, saline solution, acetic acid, and citric acid monohydrate.

14. The method of claim 8, wherein the composition consists essentially of 11.2% (w/w) iron (III) sulfate, 8.7% (w/w) calcium phosphate dibasic, 10.7% (w/w) calcium phosphate tribasic, 15.2% (w/w) hydroxyapatite, 4.5% (w/w) beta-tricalcium phosphate, 5.8% (w/w) sodium phosphate dibasic, 13.5% (w/w) sodium carbonate, 4.5% (w/w) calcium oxide, 4.5% (w/w) magnesium oxide, 1.1% (w/w) L-ascorbic acid, 20.2% powdered type-I collagen, 0.9% saline solution, 5% (v/v) acetic acid, 0.9% (wt) citric acid monohydrate, and 4-mg/mL of naloxone hydrochloride.

15.-17. (canceled)


SEQUENCE LISTING (4 sequences)

SEQ ID NO: 1 - protein, 5 aa, synthetic construct
YGGFM

SEQ ID NO: 2 - protein, 5 aa, synthetic construct
YGGFL

SEQ ID NO: 3 - protein, 8 aa, synthetic construct
  SITE 1: note = D-amino acid
  SITE 4: note = D-amino acid
  SITE 7: note = Penicillamine
  SITE 8: note = Amine modified residue
FCYWKTXT

SEQ ID NO: 4 - protein, 50 aa, synthetic construct
QSALDYFMFAVRCRHQRRQLVHFAWEHFRPRCKFVWGPQDKLRRFKPSSL